(19) United States
(12) Patent Application Publication
(10) Pub. No.: US 2026/0027346 A1
(43) Pub. Date: Jan. 29, 2026

(54) MEDICAL FUNCTIONAL UNIT COMPRISING ONE OR MORE RECHARGEABLE SODIUM-ION BATTERIES

(71) Applicant: Berlin Heart GmbH, Berlin (DE)

(72) Inventors: Helmut Jahne, Berlin (DE); Valentin Bykov, Berlin (DE); Oliver Peters, Berlin (DE)

(73) Assignee: Berlin Heart GmbH, Berlin (DE)

(21) Appl. No.: 19/283,498

(22) Filed: Jul. 29, 2025

(30) Foreign Application Priority Data
Jul. 29, 2024 (EP) 24 191 558.6

(51) Int. Cl. A61M 60/876; H01M 10/04; H01M 50/204; H01M 50/253; H01M 50/50

(52) U.S. Cl. CPC A61M 60/876; H01M 10/0422; H01M 50/204; H01M 50/253; H01M 50/50

(57) ABSTRACT

Medical functional units and methods are disclosed. A medical functional unit may comprise an electrical energy supply system which includes one or more rechargeable sodium-ion batteries, where the energy supply system and the rechargeable sodium-ion batteries comprise fewer than six, fewer than five, fewer than four, fewer than three, or fewer than two of the following six protective devices: (a) charging current protection, (b) load current protection, (c) low-voltage protection, (d) overcharge protection, (e) high temperature protection, and (f) temperature-dependent load current limitation. A method for sterilizing a functional unit may comprise rechargeable sodium-ion batteries.

### CROSS-REFERENCE TO RELATED APPLICATION

This application claims priority to EP Application No. 24 191 558.6, filed on Jul. 29, 2024, the entire content incorporated herein by reference.

### BRIEF DESCRIPTION OF THE DRAWINGS

Exemplary embodiments are shown and described below with reference to the figures.

FIG. 1 schematically shows a control device of a cardiac support system according to the prior art.

FIG. 2 shows an example of a control device of a cardiac support system according to an aspect of the invention.

### DETAILED DESCRIPTION

The invention resides in the electrical engineering and medical technology fields and can be employed particularly advantageously in portable medical technology systems, for example in portable cardiac support systems.

Thus far, batteries, in particular rechargeable batteries/accumulators, having the highest possible energy density have been employed in electronic devices, in particular in portable devices in the medical technology field, and most particularly in portable cardiac support systems. A high energy density of the batteries that are employed means that the energy required for operating the electronic devices is made available from a small volume and/or from a power supply unit having a low weight. This results in small devices and a long battery operating time. Thus far, in electronic medical devices in particular relatively expensive rechargeable lithium-ion batteries have been predominantly employed.

The use of batteries having very high energy density for upscale medical devices is described, for example, in the US patent documents U.S. Pat. Nos. 8,394,009 B2 and 8,585,571 B2. These documents describe the use of batteries having high energy density in cardiac support systems in various configurations. The focus in each case lies on the use of batteries having high energy density.

The disadvantage when employing rechargeable lithium-ion batteries/accumulators in portable electronic devices is, for example, that rechargeable lithium-ion batteries generally may experience thermal runaway under certain circumstances, that is, that a chain reaction may be set in motion in warm rechargeable battery cells in that a chemical reaction starts, which further heats these cells without outside influence. Additionally, there is the risk that rechargeable lithium-ion batteries can burn and explode. So as to minimize these risks during use, several safety mechanisms/protective functions or protective devices must be provided for safely operating rechargeable lithium-ion batteries. Usually, a battery management system (BMS) takes over the majority of protective functions. The BMS protects the rechargeable lithium-ion battery against excessive rechargeable battery voltages, for example when the charging circuit is faulty, and it protects against excessive discharge and excessive charging currents, against excessive discharging currents and against short circuits, for example when the application circuit does not provide such a protection, and it interrupts the charging of the rechargeable battery cells at excessive cell temperatures. Moreover, a BMS can take over further functions, such as for example the ascertainment of the precise charge state or also the charge equalization between different rechargeable battery cells when multiple cells are connected in series.

The risk of lithium-ion cells being able to experience thermal runaway, that is, being able to set a chain reaction in motion as a result of a chemical reaction occurring in warm rechargeable battery cells which further heats this cell without outside influence, causes the usage temperatures of rechargeable lithium-ion batteries to be limited. Both the permissible temperature during the use of devices operated with rechargeable lithium-ion batteries and the permissible temperature during the charging process of the rechargeable batteries in these devices are limited. Typical limits are outlined in DIN EN 62133-2. In general, the discharging of lithium-ion cells is limited to temperatures below 45° C., and the charging is limited to temperatures below 40° C. This already considerably limits, for example, the use of portable medical devices, in particular of cardiac support devices, comprising rechargeable lithium-ion batteries outdoors under direct solar radiation and/or with body contact.

In addition, rechargeable lithium-ion batteries have a considerable capacity reduction at lower temperatures. The user of an electronic device comprising a rechargeable lithium-ion battery therefore has to tolerate a considerable reduction in the operating time when using the device in cold environments, attributable to capacity losses.

Since one of the protective functions of rechargeable lithium-ion batteries usually is that the current is limited, there is also the risk that very high peak currents, which are required in devices to ensure proper function, for example in cardiac support systems, cannot be provided when the protective function has to limit these currents to protect the rechargeable lithium-ion batteries. Limiting peak currents may result in the need to use particularly high current capable rechargeable lithium-ion batteries, which allow particularly high peak currents, at the expense of the capacity. As a result, only a low battery capacity is then available for the relevant electronic device.

In addition, rechargeable lithium-ion batteries always require a minimum rechargeable battery voltage since the cells become irreversibly damaged at levels below this minimum voltage, and the risk of self-ignition exists. This is why these rechargeable batteries must never be fully discharged, and additionally adherence to the minimum rechargeable battery voltage level must be cyclically monitored during non-use, for example during storage. This minimum voltage prevents common sterilization of rechargeable lithium-ion batteries and of devices containing such rechargeable battery cells since the sterilization is often carried out using reactive gases, which can be combustible or explosive under the influence of electrical voltages. Such rechargeable batteries must therefore be exchangeable and can only be employed outside a sterile area. Furthermore, sterile-packaged medical devices containing customary rechargeable lithium-ion batteries cannot be stored for an extended period of time since a cyclical inspection of the charge state and, if necessary, recharging are not possible, for example, in sterile packaging.

Another problem of rechargeable lithium-ion batteries is that designs having a high energy density require not only lithium, but also cobalt and nickel for production, which are resources that are not available in large quantities on earth or almost impossible to mine without environmental damage.

It is furthermore known from document WO 2016/163473 A1 that rechargeable battery cells which are not based on lithium-ion technology and use other materials can also be employed in medical devices. However, the special characteristics of individual rechargeable battery technologies are not addressed there.

Against the background of the prior art, it is the object of the present invention to create an energy supply system for medical functional units, for example as part of cardiac support systems, which at least partly avoids the drawbacks of rechargeable lithium-ion batteries. The object is achieved by the devices, systems, and methods described below.

In one aspect, the invention relates to a medical functional unit comprising an electrical energy supply system which includes one or more rechargeable sodium-ion batteries, where the energy supply system and the rechargeable sodium-ion batteries comprise fewer than six of the following six protective devices or fewer than five or fewer than four, fewer than three, or fewer than two of the following six protective devices: (a) charging current protection (definition provided at the end of this text); (b) load current protection; (c) low-voltage protection; (d) overcharge protection; (e) high temperature protection; and (f) temperature-dependent load current limitation.

The described protective devices can be defined as follows, for example:

The charging current protection is a device that limits the charging current and/or shuts off the charging current when the same is too high, that is, when a critical threshold is exceeded.

The load current protection is a device that monitors the load current and limits the same, or shuts the same off when a critical threshold is exceeded.

The low-voltage protection is a device that, if the rechargeable battery voltage drops below a minimum threshold, initiates counter-measures (for example, shutting off a load, recharging by way of an available energy source or permanently disconnecting the cells to prevent recharging) and/or outputs or stores a warning signal.

The overcharge protection is a device that monitors the cell voltage and aborts a charging process/interrupts the charging current when a voltage threshold is exceeded.

The high temperature protection is a device that monitors the cell temperature and interrupts the discharging current when a first temperature threshold is exceeded during discharging and/or that interrupts or limits the charging current when a second temperature threshold is exceeded during charging.

A temperature-dependent load current limitation is a device that monitors the temperature of the cells during discharging and limits the load current based on the temperature of the cells.

Rechargeable sodium-ion batteries do not require the described protective devices so that multiple or all of the described protective devices do not have to be provided in rechargeable sodium-ion batteries or in functional units or devices equipped with rechargeable sodium-ion batteries. This yields considerable installation space and weight savings, so that the gross storage density for energy, that is, the volume-based or mass-based energy that can be stored in a unit volume or per mass unit, taking necessary protective devices into consideration, for rechargeable sodium-ion batteries is able to compete with the gross storage density of rechargeable lithium-ion batteries, or even better.

Saving protective devices overall not only saves installation space and mass, but also costs. Moreover, simplifying the use of rechargeable batteries when using rechargeable sodium-ion batteries also ensures a higher failure safety since the number and the scope of protective devices that may be faulty can be reduced or minimized to 0.

It is a considerable advantage that rechargeable sodium-ion batteries are employed in an electronic medical device instead of rechargeable lithium-ion batteries. On a per-cell basis, rechargeable sodium-ion batteries only have approximately half the energy density compared to rechargeable lithium-ion batteries so that, generally speaking, it was expected thus far that portable electronic devices that use rechargeable sodium-ion batteries would have to be larger in size than functionally equivalent devices comprising rechargeable lithium-ion batteries. In fact, however, rechargeable sodium-ion batteries exhibit properties that fundamentally differ from rechargeable lithium-ion batteries so that dispensing with rechargeable lithium-ion batteries also allows considerable volume and weight savings to be achieved in the devices containing the same by dispensing with protective devices.

The essence of the invention is the consideration of the gross energy density of a rechargeable battery instead of the previously customary energy density of the employed rechargeable battery cells, or the net energy density.

It is not the energy density of the rechargeable battery cell that is decisive for a portable electronic device that is operated with rechargeable batteries, but the gross energy density that the rechargeable battery cell has with all the necessary protective devices/protective circuits and add-on devices thereof. In the past, this circumstance has not been taken into account.

Another advantage when using the rechargeable sodium-ion battery is that the capacity is better utilized over the permissible temperature range (−30° C. to +60° C.). Rechargeable sodium-ion batteries have a considerably lower capacity reduction at lower temperatures than rechargeable lithium-ion batteries. This represents a considerable improvement in the usability.

According to the prior art, additional functions are often provided in connection with rechargeable batteries. A battery management system is often provided, which also takes over the transmission of data from and to the rechargeable batteries. This often times allows for communication with the battery management system from a microprocessor. For example, the BMS can establish the current charge state of the rechargeable battery cells and communicate this state to a microprocessor via a communication bus. This is very helpful in the case of rechargeable battery cells having a flat characteristic charge and discharge curve, such as, for example, in the case of rechargeable lithium-ion batteries, in particular rechargeable lithium-iron-phosphate batteries, since other methods only supply very imprecise results here.

In contrast, rechargeable sodium-ion batteries have a steeper characteristic charge and discharge curve so that relatively good information can be provided about the charge state of the rechargeable battery cells solely by measuring the cell voltage.

As a result, a battery management system can often be completely dispensed with in a rechargeable sodium-ion battery. A charge equalization between the individual cells may only be provided in rechargeable battery packs/rechargeable battery combinations comprising multiple rechargeable battery cells connected in series, both in the case of rechargeable lithium-ion batteries and in the case of rechargeable sodium-ion batteries.

By eliminating a customary battery management system, the current limiting function can also be dispensed with when using rechargeable sodium-ion batteries. In this way, devices that necessitate very high peak currents for proper function, for example in cardiac support systems, can be operated without restrictions. Permissible limits of the charge or discharge current can be at least briefly exceeded in rechargeable sodium-ion batteries, without risking fire. No compromise between high current capable battery cells/rechargeable batteries and cells/rechargeable batteries having high capacity must be found for the particular application. Both properties are combined in the same rechargeable battery.

Since a battery management system can be dispensed with in a rechargeable sodium-ion battery, the communication with a processor and the corresponding plug contacts to a rechargeable battery pack are also eliminated.

The size of rechargeable batteries can be decreased when using rechargeable sodium-ion batteries compared to other rechargeable battery designs having the same capacity, or increased capacity is available at the same size.

The gross energy density and the net energy density of the cells are the same in a rechargeable sodium-ion battery since no necessary safety-relevant add-on devices are required for these rechargeable battery cells. As a result, portable electronic devices comprising rechargeable sodium-ion batteries can be smaller or have the same size while achieving the same rechargeable battery operating time, despite the larger cell geometry.

It may be provided in an advantageous preferred embodiment, for example, that the cathode or the cathodes or the anode or the anodes of the rechargeable sodium-ion batteries are directly connected or connectible to a charging voltage source in a functional unit of the described type.

A direct connection of the connectors of rechargeable sodium-ion batteries to a charging voltage source may mean that no protective device, which is electrically connected in series with the rechargeable batteries, is provided between the same and the charging device. A direct connection can also mean in this context that the connectors of the rechargeable sodium-ion batteries are connected to a charging voltage source, without including protective devices or without interconnecting or including a battery management system. This applies regardless of which protective devices are provided in the charging device itself. Inserting rechargeable sodium-ion batteries into a functional unit or an electrical device is thus associated with very little effort. For example, the charging voltage source can be connected to the application electronics, to which, in turn, the rechargeable sodium-ion batteries can be directly connected.

Moreover, it may be provided that the charging voltage source is formed by an inductive charging circuit.

In many devices that are operated with rechargeable batteries, charging is possible by inductive charging via a magnetic field without establishing a galvanic connection. This may be particularly attractive for implantable functional units in medical technology since no transcutaneous connection to the cells to be charged is necessary when charging inductively, and the risks of infections due to transcutaneous connections can thus be minimized.

It may also be provided that, in a functional unit of the described type, the cathode or the cathodes and the anode or the anodes of the rechargeable sodium-ion batteries are directly connected or connectible to an electrical unit which can be operated using the energy from the rechargeable sodium-ion battery or batteries.

A direct connection of the connectors of the rechargeable sodium-ion batteries to an electrical unit which can be operated using the energy from the rechargeable sodium-ion battery or batteries can mean that no protective device, which is electrically connected in series with the rechargeable batteries, is provided between the same and the electrical unit. This applies regardless of which protective devices are provided in the electrical unit itself. A direct connection can also mean in this context that the connectors of the rechargeable sodium-ion batteries are connected to an electrical unit, without including protective devices or without interconnecting or including a battery management system. Inserting rechargeable sodium-ion batteries into a functional unit or an electrical device is thus associated with very little effort.

Another preferred embodiment may provide, for example, that the energy supply system and the rechargeable sodium-ion batteries in a functional unit of the described type do not comprise any of the following six protective devices: (a) charging current protection, (b) load current protection, (c) low-voltage protection, (d) overcharge protection, (e) high temperature protection, and (f) temperature-dependent load current limitation; and that the cathode or the cathodes and the anode or the anodes of the rechargeable sodium-ion batteries are directly connected or connectible to a charging voltage source.

In addition, it shall be noted that the charging voltage source can also be formed in this case by an inductive charging circuit in some instances.

It may also be provided in a functional unit of the described type that the energy supply system and the rechargeable sodium-ion batteries do not comprise any of the following six protective devices: (a) charging current protection, (b) load current protection, (c) low-voltage protection, (d) overcharge protection, (e) high temperature protection, and (f) temperature-dependent load current limitation; and that the cathode or the cathodes and the anode or the anodes of the rechargeable sodium-ion batteries are directly connected or connectible to an electrical unit which can be operated using energy from the rechargeable sodium-ion battery or batteries.

The last two preferred embodiments make it clear that a functional unit comprising rechargeable sodium-ion batteries not only can make do with fewer than six of the described protective devices, but also entirely without any of the described protective devices, and the rechargeable sodium-ion batteries can be easily integrated into the functional unit and be connected thereto. As indicated above, this is also possible when none of the described protective devices is provided otherwise in the functional unit.

Another preferred embodiment may also be provide in a functional unit of the described type that the energy supply system and the rechargeable sodium-ion batteries do not comprise a protective device that detects measured variables of the rechargeable sodium-ion batteries during storage and/or outputs or stores a warning signal when a drop below a voltage threshold occurs.

As previously stated, storing rechargeable lithium-ion batteries, which thus far have been used on a large scale, is hazardous since a drop below a low-voltage threshold may occur. Below this threshold, chemical reactions may be set in motion, which can destroy the rechargeable battery by fire or explosion without outside influence, and in the process also considerably jeopardize other units and the surrounding area. For this reason, rechargeable lithium-ion batteries must be monitored, for example, with respect to the cell voltage during storage. Monitoring devices are known for this purpose, which output an alarm signal as soon as the voltage of a cell drops below a low-voltage threshold. The prerequisite for reliable storage is that counter-measures can also be taken in response to a warning signal.

The described low-voltage problem and a corresponding risk do not occur when storing rechargeable sodium-ion batteries so that rechargeable sodium-ion batteries can be stored in a simple manner without great effort and without protective devices and monitoring devices. This circumstance makes it possible to store and distribute rechargeable sodium-ion batteries regardless of time schedules, even if the rechargeable batteries, for example, have already been installed in packaged functional units and cannot be recharged.

In a further preferred embodiment, it may be provided that the medical functional unit is part of a ventricular assist device (VAD).

Ventricular assist devices and systems require an extremely reliable and fail-safe energy supply system since a failure of the supply system immediately jeopardizes the health of a patient. At the same time, however, such cardiac support devices are to limit the mobility of the patients as little as possible and should therefore have the smallest and most lightweight design possible. A high gross energy density of the rechargeable batteries required for mobile energy supply, including the necessary protective devices, combined with a high failure safety due to an intrinsic safety of the rechargeable battery cells is thus extremely valuable, especially for cardiac support devices.

Moreover, it may be provided that the ventricular assist device comprises an implantable part and an extracorporeal part, and that the rechargeable sodium-ion batteries are arranged in the extracorporeal part or that the rechargeable sodium-ion batteries are arranged in the implantable part.

It may be advantageous in the individual case for rechargeable sodium-ion batteries to be arranged in the extracorporeal part of a cardiac support device. In this part, they can be easily recharged and are easily accessible.

In other instances, it may also be useful to utilize rechargeable sodium-ion batteries in the implantable or implanted part of cardiac support devices, for example when an implanted blood pump is to be driven by way of the energy stored in the rechargeable batteries.

It may also be provided that the rechargeable sodium-ion batteries can be connected to the electrical part of the medical functional unit by means of one or more plug connectors or that the rechargeable sodium-ion batteries can be inserted into a battery compartment of the medical functional unit and contacted therein by means of spring-loaded pressure contacts.

Since the rechargeable sodium-ion battery or batteries can be used without further protective devices, these can be easily inserted into a functional unit or a device. Since they can be completely discharged without risk, they can also be sterilized easily and without major effort and be inserted into a functional unit in the sterile state; for this, simple contacting is helpful because the risk of contamination is minimized during the insertion of the rechargeable batteries. In many instances, however, it is also possible to sterilize the rechargeable batteries together with a functional unit and/or a device.

Another implementation option may provide for the rechargeable sodium-ion batteries to be fixedly installed in a housing of the medical functional unit.

The property of the rechargeable batteries of being fixedly installed can mean, for example, that these are installed in a housing so as not to be dismountable from the housing or removable from the housing without special tools/without a tool that goes beyond a screwdriver or so as to be dismountable from the housing or removable from the housing only by trained staff/only by destroying integral bonds or so as not to be reversibly be dismountable from the housing or removable from the housing. Fixedly installed rechargeable battery cells have the advantage that no plug connectors are necessary and potential openings for replacing the rechargeable batteries can be dispensed with. This increases the reliability since plug contacts or the interior space of the medical device can become contaminated by openings and can later corrode. A fixed installation of the power supply system makes a hermetically sealed housing without openings possible.

Such an integration of the rechargeable batteries into a functional unit is possible, amongst others, because longer storage of the functional unit, together with the rechargeable batteries, is possible without monitoring. In addition, the rechargeable batteries can be sterilized together with the functional unit for medical applications since the rechargeable batteries can be completely discharged beforehand without risk. A complete discharge of the rechargeable batteries in the present context can mean that these are stored in a short-circuited state over an extended period of time, that is, over days or weeks, and no measurable residual voltage is present.

The invention also relates to a medical functional unit of the type described above, for example a control device for a cardiac support device, comprising sterile packaging that encloses the functional unit together with one or more rechargeable sodium-ion batteries.

This combination is not useful in the case of rechargeable lithium-ion batteries because these must be continuously monitored and recharged when a low-voltage threshold is reached. The use of rechargeable sodium-ion batteries requires neither monitoring nor recharging so that the functional unit, for example in the form of a control device of a cardiac support system, can be stored and transported in the sterile packaging over an extended period of time without difficulty. For example, the sterile packaging can be a hermetically welded flexible plastic pouch.

The invention can also relate to a ventricular assist system comprising a medical functional unit of the above-described type.

As described above, a functional unit, for example a control unit or an energy supply system for a pump drive, can then be equipped with rechargeable sodium-ion batteries in an implantable or an extracorporeal part of the cardiac support system, for example.

In addition to a functional unit of the above-described type and a ventricular assist system, the invention also relates to a method for sterilizing one or more rechargeable sodium-ion batteries, characterized in that the rechargeable sodium-ion battery or batteries is or are initially completely discharged and sterilized after having been discharged.

Sterilization is a very complex process in rechargeable batteries that have thus far been customary for portable devices, for example, because this has to be carried out when a residual voltage is present in the rechargeable batteries. Some sterilization methods also require heating of the rechargeable batteries. For this reason, a number of sterilization methods are not an option for many types of rechargeable batteries. The possibilities are endless when it comes to the type of sterilization for rechargeable sodium-ion batteries because they are robust, tolerate high temperatures, and can be completely discharged.

It may also be provided in a method for the described type that, for the sterilization of a functional unit comprising one or more rechargeable sodium-ion batteries, the rechargeable sodium-ion batteries, with which the functional unit is being operated, are completely discharged before or after insertion into the functional unit, and that the functional unit is sterilized together with the rechargeable sodium-ion batteries after the rechargeable sodium-ion batteries have been discharged.

It is thus possible, with little effort, to sterilize the functional unit in a sterilization method together with the rechargeable batteries, wherein the rechargeable batteries can be discharged beforehand in the or outside the functional unit.

It may also be provided in a method of the above-described type that, for the sterilization of an, in particular implantable, medical functional unit, the rechargeable sodium-ion batteries, with which the functional unit is being operated, are completely discharged before or after insertion or fixed installation into the medical functional unit, and that the medical functional unit is sterilized together with the rechargeable sodium-ion batteries after the rechargeable sodium-ion batteries have been discharged.

The above-described sterilization process unfolds many of its advantages in particular during the sterilization of medical functional units which come in contact with patients or are even entirely or partly implanted.

An electronic device can be sterilized with installed rechargeable sodium-ion batteries, and it may be stored for years in sterile packaging without cyclical monitoring. Both aspects are only possible because a complete discharge of rechargeable sodium-ion battery cells is possible, without the rechargeable battery cells incurring damage as a result. This allows, for example, the equipping with rechargeable sodium-ion batteries already during the electronics production process.

Thus far, it was customary for cardiac support systems during heart surgery to supply electronic devices in the sterile area with electrical current via cables from other energy sources outside the sterile area. It was only possible to use a non-sterile rechargeable battery after successful surgery. For this purpose, an opening for introducing a rechargeable battery had to be provided in the housing of the relevant electronic device. This housing opening had to be designed so as to be closable in a liquid-tight manner. The additionally required installation space for an opening that can be closed in a liquid-tight manner can be saved by using rechargeable sodium-ion batteries.

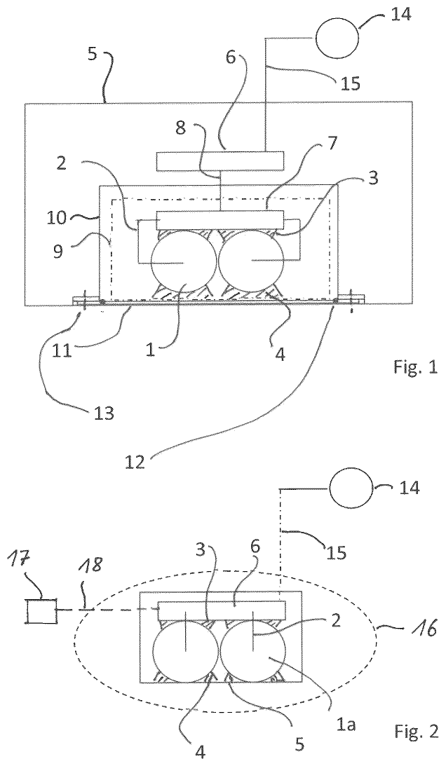

According to the prior art corresponding to FIG. 1, the planning for a cardiac support system, for example a ventricular assist device (VAD) system, is as follows: A portable control unit comprising a housing 5 is provided. This control unit can control a symbolically illustrated blood pump 14 via a line 15 and supply it with the necessary energy. The blood pump can be implanted, wherein the control unit can be provided both so as to be implantable and extracorporeal. So as to achieve the planned operating time, four rechargeable lithium-ion batteries 1 having high energy density, in the 18650 format, are provided, wherein each cell is to have a capacity of 3000 mAh at a typical cell voltage of 3.6 V. In the functional unit designed as the control unit, a battery management system 7 measuring 65 mm×72 mm and having a thickness of 3 mm is provided for these four rechargeable lithium-ion batteries within a rechargeable battery pack and the encasement 9 thereof. The four rechargeable battery cells are integrated together with the battery management system in the rechargeable battery pack with a rechargeable battery pack housing or an encasement 9, which has a size of approximately 74 mm×68 mm and a thickness of 23 mm. Within the rechargeable battery pack, electrical connectors of the rechargeable battery cells are connected via connecting lines 2 to the battery management system 7. This rechargeable battery pack is provided with a pigtail 8 comprising a plug connector, which is only shown symbolically and by way of which it is contacted on the main circuit board of the application electronics 6 of the control device. So as to be able to insert the rechargeable battery pack into the housing 5 of the control unit of the VAD system, a rechargeable battery compartment 10 is provided in the housing, which is specially produced for this purpose and which has a cover 11 that can be closed in a waterproof manner and has corresponding seals 12 and fastening elements 13. It is possible, for example, for screws having an unusual head to be provided as fastening elements, which cannot be loosened by way of a commercially available screwdriver. A recess having a polygonal cross-section may be suitable for this purpose, for which a matching specialty tool is only supplied to a select group of people.

The rechargeable battery pack comprising the described rechargeable lithium-ion batteries cannot be sterilized. The rechargeable lithium-ion battery comprising four rechargeable battery cells therefore has to be designed so as to be able to be installed into the control unit, which up until then is sterile, after the heart surgery for implanting the heart pump. The rechargeable battery compartment 10 requires a volume of approximately 78 mm×72 mm×27 mm in the housing 5 of the control unit, in addition to the mounting site for the cable to the battery management system. In addition, space is required for the cover, which can be removed and is surrounded by a waterproof seal. The rechargeable battery pack can be contacted in the battery compartment by springs provided there. All in all, this results in a necessary volume in the housing of approximately 170 cm3 according to the prior art. The intermediate space between the cylindrical rechargeable battery cells cannot be used for the electronics of the control unit/application electronics because it is separated by multiple intermediate walls from the electronics on the main circuit board of the application electronics.

The rechargeable battery cells 1 can be bonded to the battery management system 7 by means of an adhesive bond 3. Likewise, the rechargeable battery cells 1 can also be connected by an adhesive bond 4 to the encasement 9 of the rechargeable battery pack.

The implementation according to an aspect of the invention of the functionally equivalent cardiac support system is to be described based on exactly this example in FIG. 2, wherein functionally equivalent units are denoted by identical reference numerals in the two figures.

So as to achieve the same rechargeable battery operating time as with the above-described rechargeable lithium-ion batteries, four rechargeable sodium-ion batteries 1a having the size 26700, each having 3300 mAh and a typical cell voltage of 3.1 V, are selected. Since a deep discharge is permissible for the rechargeable sodium-ion batteries, these can be completely discharged again after the initial operation in the manufacturer's plant. They can then be subsequently sterilized within the control unit and remain in the sterile packaging of the control unit or of the cardiac support system for years without monitoring. FIG. 2 with dotted lines symbolically shows the sterile packaging 16, which can take on the form of a hermetically sealed plastic pouch. During charging and during active use of the functional unit and of the rechargeable batteries, the sterile packaging is generally no longer present. During initial use, the rechargeable sodium-ion batteries are charged and are then available ready for use. The typical service life of the rechargeable sodium-ion batteries is 10 to 15 years. The typical period of use of a control unit for a VAD system is 3 years. As a result, there is no need to ever replace the rechargeable sodium-ion battery within the operating life of the control unit. The rechargeable sodium-ion battery unit composed of four cells does not require a battery management system. The individual rechargeable sodium-ion battery cells 1a can be directly mounted with the electrical connectors 2 thereof in the form of solder lugs on the main circuit board, which forms the application electronics 6 or is part of the application electronics/of the control device. The intermediate space between the rechargeable battery round cells 1a can also be partly utilized, either for electronics on the main circuit board or for reinforcing ribs in the housing. FIG. 2 shows that this intermediate space between the rechargeable battery cells is utilized by adhesive bonds 3, 4, so as to provide mechanical relief to the solder spots between the rechargeable battery and the application electronics on the one hand, and increase the mechanical rigidity of the housing by adhesive bonds between the rechargeable batteries and the housing 5 on the other hand. The four sodium-ion round cells having the 26700 format jointly occupy a volume of 149 cm3 in the control unit.

As is shown in FIG. 2, a connection of the rechargeable sodium-ion batteries to a charging voltage source 17 can be established via the printed circuit board of the application electronics and an electrical connector. This printed circuit board can comprise connectors that can be connected to the external charging voltage source, for example by means of a plug connection 18 or by means of an inductive charging circuit. The application electronics can comprise a charge controller, which is connected to the rechargeable sodium-ion batteries without interposed protective devices. Protective devices are not provided on the printed circuit board for this purpose. The connecting line/plug connection 18 is shown by dashed lines in FIG. 2 since the charging connection is generally only utilized when the sterile packing 16 has been opened or removed.

Despite the lower net energy density of the rechargeable sodium-ion battery cells compared to the rechargeable lithium-ion battery cells, in sum the use of rechargeable sodium-ion battery cells thus results in a volume reduction in the portable control device forming a medical functional unit. Similarly, the weight is also reduced.

Since the control device comprising the rechargeable sodium-ion batteries in sterilized form can be easily stored and transported, this also results in a considerable simplification when it comes to the logistics.

It has also proven to be particularly advantageous that the raw materials required for creating rechargeable sodium-ion batteries are essentially available boundlessly. Sodium, in the form of sodium chloride, is essentially available in unlimited quantity in the oceans. The environmental burden is also significantly decreased when using rechargeable sodium-ion batteries.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one or more element alone or the one or more element in combination with one or more of the other elements which may also include, in combination, additional elements not listed. Unless otherwise indicated or the context suggests otherwise, as used herein, “a” or “an” means “at least one” or “one or more.”

1. A medical functional unit comprising:
an electrical energy supply system including one or more rechargeable sodium-ion batteries;
where the energy supply system and the rechargeable sodium-ion batteries comprise fewer than six, or fewer than five, or fewer than four, or fewer than three, or fewer than two of the following six protective devices:
charging current protection;
load current protection;
low-voltage protection;
overcharge protection;
high temperature protection; and
temperature-dependent load current limitation.

2. The medical functional unit of claim 1, where a cathode and an anode of the rechargeable sodium-ion batteries are directly connectible to a charging voltage source.

3. The medical functional unit of claim 2, where the charging voltage source is formed by an inductive charging circuit.

4. The medical functional unit of claim 1, where the cathode and the anode are directly connectible to an electrical unit operable using energy from the one or more rechargeable sodium-ion batteries.

5. The medical functional unit of claim 1, where the energy supply system and the rechargeable sodium-ion batteries comprise none of the following six protective devices:
charging current protection;
load current protection;
low-voltage protection;
overcharge protection;
high temperature protection; and
temperature-dependent load current limitation; and
where a cathode and an anode of the rechargeable sodium-ion batteries are directly connectible to a charging voltage source.

6. The medical functional unit of claim 1, where the energy supply system and the rechargeable sodium-ion batteries comprise none of the following six protective devices:
charging current protection;
load current protection;
low-voltage protection;
overcharge protection;
high temperature protection; and
temperature-dependent load current limitation; and
where a cathode and an anode of the rechargeable sodium-ion batteries are directly connectible to an electrical unit operable using energy from the one or more rechargeable sodium-ion batteries.

7. The medical functional unit of claim 1, where the energy supply system and the rechargeable sodium-ion batteries comprise no protective device that detects measured variables of the rechargeable sodium-ion batteries during storage and/or outputs or stores a warning signal when a drop below a voltage threshold occurs and/or that the medical functional unit is part of a ventricular assist device.

8. The medical functional unit of claim 7, where the medical functional unit is part of a ventricular assist device comprising an implantable part and an extracorporeal part, and where the rechargeable sodium-ion batteries are arranged in the extracorporeal part or in the implantable part.

9. The medical functional unit of claim 1, where the rechargeable sodium-ion batteries are connectible to an electrical part of the medical functional unit by means of one or more plug connectors (8) or where the rechargeable sodium-ion batteries are insertable into a battery compartment of the medical functional unit and contacted therein by spring-loaded pressure contacts.

10. The medical functional of claim 1, where the rechargeable sodium-ion batteries are fixedly installed in a housing of the medical functional unit.

11. The medical functional unit of claim 1, comprising sterile packaging that encloses the functional unit together with the one or more rechargeable sodium-ion batteries.

12. A ventricular assist system comprising a medical functional unit of claim 1.

13. A method for sterilizing one or more rechargeable sodium-ion batteries, characterized in that the rechargeable sodium-ion battery or batteries is or are initially completely discharged and sterilized after having been discharged.

14. The method of claim 13, characterized in that, for the sterilization of a functional unit comprising one or more rechargeable sodium-ion batteries, the rechargeable sodium-ion batteries, with which the functional unit is being operated, are completely discharged before or after insertion into the functional unit, and that the functional unit is sterilized together with the rechargeable sodium-ion batteries after the rechargeable sodium-ion batteries have been discharged.

15. The method of claim 13, characterized in that, for the sterilization of an, in particular implantable, medical functional unit, the rechargeable sodium-ion batteries, with which the functional unit is being operated, are completely discharged before or after insertion or fixed installation into the medical functional unit, and that the medical functional unit is sterilized together with the rechargeable sodium-ion batteries after the rechargeable sodium-ion batteries have been discharged.
